(19) United States
(12) Patent Application Publication
(10) Pub. No.: US 2018/0344179 A1
(43) Pub. Date: Dec. 6, 2018

(54) METHODS AND APPARATUS FOR DETECTING VITAL SIGNS DURING CARDIOPULMONARY RESUSCITATION

(71) Applicant: KONINKLIJKE PHILIPS N.V., EINDHOVEN (NL)

(72) Inventor: Ihor Olehovych KIRENKO, VELDHOVEN (NL)

(21) Appl. No.: 15/780,662

(22) PCT Filed: Dec. 1, 2016
(86) PCT No.: PCT/EP2016/079358

§ 371 Date: Jun. 1, 2018

(30) Foreign Application Priority Data
Dec. 15, 2015 (EP) 15199986.9

(51) Int. Cl. A61B 5/024; A61B 5/00; A61H 31/00; A61B 5/1455; A61M 16/00

(52) U.S. Cl. CPC A61B 5/02416; A61B 5/6822; A61H 31/008; A61B 5/14551; A61M 16/0048; A61B 2505/01; A61H 2201/501; A61B 5/6887; A61H 2201/5028; A61H 2230/06; A61H 2201/5061; A61B 5/721; A61H 2201/1609; A61B 5/6843

(57) ABSTRACT

In various embodiments, an apparatus to aid in cardiopulmonary resuscitation (“CPR”), may include: a support structure (200, 300, 400) shaped to support a head and neck of a patient at respective positions suitable for opening an airflow of the patient during performance of CPR on the patient; one or more electromagnetic radiation sources (208, 308, 408) mounted on the support structure to emit electromagnetic radiation having a wavelength within a predetermined frequency range of wavelengths onto skin of the patient; and one or more electromagnetic or optical sensors (210, 310, 410) mounted on the support structure to detect electromagnetic radiation within the predetermined frequency range that is reflected from or transmitted into the patient's skin.

### FIELD OF THE INVENTION

The present invention is directed generally to health care. More particularly, various inventive methods and apparatus disclosed herein relate to detecting one or more of a patient's vital signs during performance of cardiopulmonary resuscitation (“CPR”).

### BACKGROUND OF THE INVENTION

Cardiopulmonary resuscitation (“CPR”) is an emergency procedure that is performed on a patient under cardiac arrest in an effort to create artificial circulation, namely, by manually pumping blood through the heart. CPR may be performed until further measures are taken to restore spontaneous blood circulation and breathing. CPR techniques may involve performing chest compressions of various magnitudes, such as at least 5 cm (2 in) deep, at various intervals, such as at least one hundred per minute. In some instances, CPR may be stopped when return of spontaneous circulation (“ROSC”) is detected. Various contact sensors such as photoplethysmogram (“PPG”) sensors may be used to detect a variety of vital signs, including but not limited to absence/presence of the patient's pulse, arterial blood oxygen saturation (“SpO₂”), tissue oxygen saturation, perfusion, etc., as well as to monitor the patient's pulse rate during CPR. However, it is difficult to non-invasively detect ROSC while chest compressions are being performed. Interrupting chest compressions to accurately detect ROSC may increase the likelihood of a negative outcome. Thus, there is a need in the art to provide techniques and apparatus to aid in detection and/or monitoring of a patient's vital signs, such as pulse, pulse rate variability, SpO₂, and/or tissue oxygenation (StO₂) during performance of CPR.

### SUMMARY OF THE INVENTION

The present disclosure is directed to inventive methods and apparatus for detecting one or more of a patient's vital signs during performance of CPR. A head and/or neck support structure such as a specially-shaped pillow may be used to position a patient's head and neck in respective positions suitable for opening an airflow of the patient during performance of CPR on the patient. The support structure may include one or more electromagnetic radiation sources such as one or more light-emitting diodes to emit electromagnetic radiation (e.g., light) within a particular frequency range of wavelengths onto the patient's skin, e.g., at or near the neck. The support structure may further include one or more electromagnetic or optical sensors such as one or more light sensors to detect electromagnetic radiation within the particular frequency range of wavelengths, and particularly to detect light emitted by the one or more sources, e.g., as it transmits into/through or is reflected from the patient's skin. Based on the detected electromagnetic radiation, logic integral with or separate from the support structure may determine one or more vital signs of the patient, such as pulse and/or SpO₂. In various embodiments, the sources and/or sensors may not physically contact the patient's skin, thereby eliminating or at least reducing “noise” resulting from chest compressions performed during CPR.

Generally, in one aspect, an apparatus to aid in CPR may include: a support structure shaped to support a head and neck of a patient at respective positions suitable for opening an airflow of the patient during performance of CPR on the patient; one or more electromagnetic radiation sources mounted on the support structure to emit electromagnetic radiation having a wavelength within a predetermined frequency range of wavelengths onto skin of the patient; and one or more electromagnetic or optical sensors mounted on the support structure to detect electromagnetic radiation within the predetermined frequency range that is reflected from or transmitted into the patient's skin.

In various embodiments, the one or more electromagnetic or optical sensors may be mounted on the support structure in a manner selected to avoid physical contact with the patient's skin. In various versions, the one or more electromagnetic or optical sensors may be mounted within recesses of the support structure. In various versions, the one or more electromagnetic or optical sensors may be mounted at positions on a surface of the support structure that do not come into physical contact with the patient's skin when the patient's head and neck are properly supported by the support structure. In various versions, the one or more electromagnetic or optical sensors may be mounted within a neck cradle of the support structure. In various versions, the one or more electromagnetic or optical sensors may be mounted at one or more sides of the neck cradle.

In various embodiments, the one or more electromagnetic radiation sources may be mounted on the support structure in a manner selected to avoid physical contact with the patient's skin. In various embodiments, at least one of the one or more electromagnetic radiation sources may surround at least one of the one or more electromagnetic or optical sensors. In various embodiments, the one or more electromagnetic radiation sources may be spaced from the one or more electromagnetic or optical sensors. In various embodiments, the electromagnetic or optical sensors may emit modulated electromagnetic radiation. In various embodiments, the apparatus may further include logic operably coupled with the one or more electromagnetic or optical sensors and one or more pressure sensors. The logic may be configured to determine a pulse rate of the patient based on signals from both the one or more electromagnetic or optical sensors and the one or more pressure sensors.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 illustrates examples of improper and proper head and neck positioning during performance of CPR.

FIGS. 2-3 depict various apparatus configured with selected aspects of the present disclosure, in accordance with various embodiments.

FIG. 4 depicts a cross sectional view of an apparatus configured with selected aspects of the present disclosure, in accordance with various embodiments.

FIG. 5 depicts an example method, in accordance with various embodiments.

### DETAILED DESCRIPTION OF EMBODIMENTS

Various sensors such as PPG sensors may be used to detect patient vital signs, such as absence/presence of the patient's pulse and/or SpO₂, as well as to monitor the patient's pulse rate during CPR. However, CPR techniques may require that chest compressions be performed. Movement of the patient's body resulting from chest compressions may make non-invasive detection of reliable ROSC difficult, and interrupting chest compressions to accurately detect ROSC may increase the likelihood of a negative outcome. Thus, Applicants have recognized and appreciated that it would be beneficial to provide techniques and apparatus to aid in detection and/or monitoring of a patient's vital signs during performance of CPR. In view of the foregoing, various embodiments and implementations of the present invention are directed to apparatus and methods for detecting a patient's vital signs during performance of CPR.

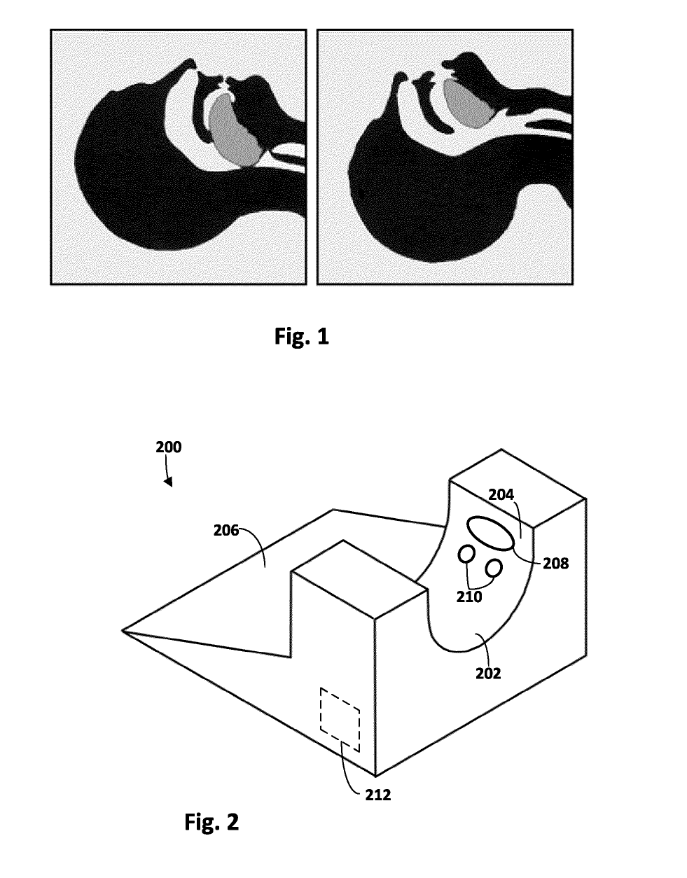

FIG. 1 depicts examples of improper and proper position of a patient's head and neck during CPR. One the left, the patient's head is more or less aligned with the patient's neck. This positioning makes it more likely that the patient's tongue (shown in gray) will contact the back of the patient's throat, blocking the patient's airway. One the right, the patient's head and neck have been subjected to the so-called “head-tilt/chin-lift” maneuver in which the patient's head is tilted back (and the patient's chin lifted). This proper positioning spaces the patient's tongue from the back of the patient's throat, thereby opening the patient's airway.

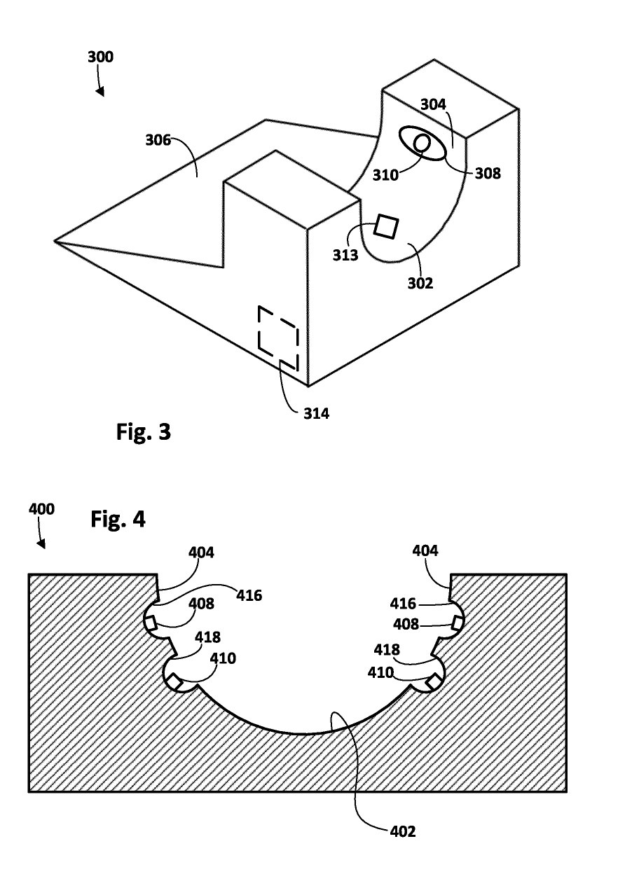

FIG. 2 depicts a head and/or neck support structure 200 (alternatively referred to simply as “support structure”) that is usable to properly position a patient's head and/or neck during performance of CPR, as is depicted in FIG. 1. Support structure 200 may be constructed with various materials and may have various levels of stiffness. In some embodiments, support structure 200 may be a pre-formed pillow, e.g., created at least in part with various types of polymers, foam, and/or rubber. In other embodiments, support structure 200 may include other (undepicted) mechanical features, such as legs (may be adjustable), restraints, and so forth.

In various embodiments, support structure 200 may include a neck cradle 202 (or simply “cradle”) that is shaped to support the neck (not depicted) of a patient (also not depicted). Cradle 202 may include one or more interior sides 204 that may or may not contact the patient's neck when placed in cradle 202. The patient's upper back or shoulders may rest on an optional back support section 206 when the patient's neck is supported in cradle 202. The patient's head may hang from the opposite side of cradle 202 from back support section 206, so that the patient's head is tilted backwards (and the patient's chin lifted upwards) by the force of gravity. As described above, positioning the neck and head in such a manner may remove the patient's tongue from the back of the patient's throat, opening the patient's airways.

In various embodiments, support structure 200 may include a variety of sensors and other components configured to detect and/or monitor one or more of the patient's vital signs in a non-invasive manner that is not affected by movement of the patient's body caused by, for instance, chest compressions performed during CPR. For example, in FIG. 2, support structure includes one or more electromagnetic radiation sources 208 and one or more electromagnetic or optical sensors 210. These components may be operably coupled with logic 212 so that together, they may detect and/or monitor the patient's pulse.

The one or more electromagnetic radiation sources 208 may come in various forms. In some embodiments, an electromagnetic radiation source 208 may come in the form of one or more light sources, such as one or more light-emitting diodes (“LED”). Each electromagnetic radiation source may emit electromagnetic radiation within various frequency ranges, such as various sub-ranges within the visible and/or invisible (e.g., infrared) spectrums (i.e., visible or invisible light). Electromagnetic radiation sources 208 may be positioned on support structure 200 so that they may emit electromagnetic radiation towards the patient's skin. For instance, in FIG. 2, they are positioned in cradle 202 so that they emit radiation toward the side of the patient's neck. The one or more electromagnetic or optical sensors 210 likewise may come in various forms that are adapted to detect electromagnetic radiation within a frequency range that at least partially overlaps the predetermined frequency range emitted by the one or more electromagnetic radiation sources 208. For example, electromagnetic or optical sensors 210 may include but are not limited to various forms of photodetectors, such as cameras, active-pixel sensors, reverse-biased LEDs, photodiodes, photoresistors, photovoltaic cells, phototransistors, and so forth.

Electromagnetic radiation that is emitted towards the patient's skin by electromagnetic radiation sources 208 may be reflected from and/or transmitted into the patient's skin. This reflected and/or transmitted electromagnetic radiation may be detected by electromagnetic or optical sensors 210. Electromagnetic or optical sensors 210 may provide one or more signals of the detected electromagnetic radiation to logic 212. Based on the received signals, in various embodiments, logic 212 may determine one or more vital signs of the patient, such as pulse rate and/or SpO₂.

In various embodiments, electromagnetic radiation sources 208 and/or sensors 210 may be mounted on support structure 200 in a manner selected to avoid physical contact with the patient's skin. This may reduce or eliminate noise produced by movement of the patient caused by, for instance, chest compressions. For example, in some embodiments, electromagnetic radiation sources 208 and/or sensors 210 may be mounted within recesses of the support structure, as will be described below with reference to FIG. 4.

In some embodiments, electromagnetic radiation sources 208 and/or sensors 210 may be mounted at positions on a surface of support structure 200 that do not come into physical contact with the patient's skin when the patient's head and neck are properly supported by support structure 200. For example, in some embodiments, electromagnetic radiation sources 208 and/or sensors 210 may be mounted within cradle 202 of support structure 200, e.g., at the interior sides 204 which may not physically contact the sides of the patient's neck. Thus, in some embodiments, cradle 202 may be sufficiently wide so that sides 204 do not contact most or all patients' necks.

Logic 212 may take various forms. In some embodiments, logic 212 may include one or more microprocessors operably coupled with memory (not depicted) storing instructions that, when executed, cause logic 212 to perform various operations described herein. In other embodiments, logic 212 may come in the form of an application-specific integrated circuit (“ASIC”) or field-programmable gate array (“FPGA”).

Not every embodiment may include integral logic 212. For example, FIG. 3 depicts an alternative embodiment of a support structure 300 configured with selected aspects of the present disclosure. FIG. 3 may include components that are similar to those of FIG. 2, except they start with a “3” instead of a “2”. In FIG. 3, a communication interface 314 is provided instead of logic. The communication interface 314 may be configured to provide, e.g., to a remote computing device (not depicted) over one or more networks (not depicted), signals indicative of the patient's pulse that is detected by electromagnetic or optical sensors 310. In other embodiments, a support structure may include both logic and a communication interface. In some such embodiments, the logic may determine a pulse rate of the patient based on signals provided by electromagnetic or optical sensors mounted on the support structure, and then may provide output indicative of this determination through the communication interface to a remote computing device. Communication interface 314 may come in various forms and may employ various wired or wireless technologies to communication with a remote computing device, including but not limited to Bluetooth, Ethernet, Wi-Fi, universal serial bus (“USB”), serial, PCIe, a proprietary scheme, and so forth.

The embodiment of FIG. 3 is different than that depicted in FIG. 2 in other ways as well. For example, in FIG. 3, electromagnetic radiation source 308 surrounds electromagnetic or optical sensor 310. This may be particularly well-suited for detecting radiation that is reflected from the patient's skin. By contrast, in the embodiment depicted in FIG. 2, electromagnetic or optical sensor 210 is spaced from electromagnetic radiation source 208. This may be particularly well-suited for detecting radiation that has travelled at least partially through (i.e., transmissive) the patient's skin. For example, by being spaced from electromagnetic radiation source 208, an electromagnetic or optical sensor 210 may be at an angle relative to electromagnetic radiation source 208, and thus may be adapted to detect light that passes partially or wholly through the patient's skin. Other configurations are possible as well, such as both types of source/sensor arrangements being present simultaneously, so that both reflective and transmissive techniques may be employed.

FIG. 3 is also different in that it includes a pressure sensor 313 placed at a bottom of cradle 302. Pressure sensor 313 may detect when a patient's neck has been placed in cradle 302, e.g., to initiate measurement. In some embodiments, pressure sensor 313 may be used additionally or alternatively to detect bodily movement of the patient, e.g., that results from chest compressions. Pressure sensor 313 may produce a signal indicative of the pressure it senses and may provide that signal to logic (e.g., integral with support structure as shown in FIG. 2 or remote therefrom as shown in FIG. 3). The logic may then utilize the pressure sensor signal in conjunction with signals from electromagnetic or optical sensors 310 to more accurately determine various vital signs, such as pulse rate.

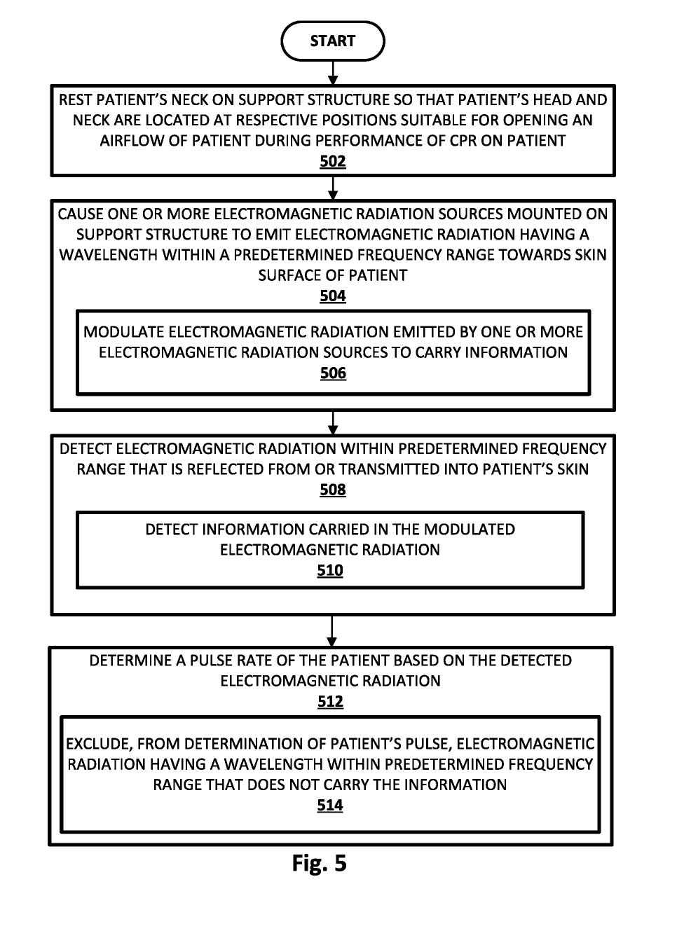

FIG. 4 depicts a cross-sectional view of a support structure 400 configured with selected aspects of the present disclosure. As was the case with FIGS. 2 and 3, various components of support structure 400 are similar to those depicted in previous figures, and thus are numbered similarly. In FIG. 4, recesses 416 and 418 are provided for electromagnetic radiation sources 408 and sensors 410, respectively. By positioning sources/sensors 408/410 within recesses 416/418, contact by the sources/sensors 408/410 may be avoided. The sizes and shapes of recesses 416/418 are for illustration only, and are not meant to be to scale, or to be limiting. FIG. 5 depicts an example method 500 that may be performed in order to detect

ROSC during performance of CPR without interference from external noise caused by, for instance, chest compressions, in accordance with various embodiments. While the operations are depicted in a particular order, this is not meant to be limiting. In various embodiments, various operations may be added, omitted, and/or reordered. At block 502, a patient's neck may be rested on a cradle (e.g., 202, 302 402) of a support structure (e.g., 200, 300, 400) configured with selected aspects of the present disclosure. This may cause the patient's head and neck to be located at respective positions suitable for opening an airflow of the patient during performance of CPR.

At block 504, one or more electromagnetic radiation sources (e.g., 208, 308, 408) mounted on the support structure may be activated (e.g., in response to a user command, user actuation of a button or switch, the weight of the patient's neck on the cradle, etc.) to emit electromagnetic radiation having a wavelength within a predetermined frequency range towards the patient's skin. In some embodiments, causing the one or more electromagnetic radiation sources to emit electromagnetic radiation may include, at optional block 506, modulating the electromagnetic radiation, e.g., to carry information. The carried information may include any sequence of characters, bits, numbers, etc., such as, for instance, a code unique to the support structure, a random sequence, a repeating pattern at a particular frequency, and so forth. In various implementations in which the electromagnetic radiation sources emit visible light, the light may be modulated at a frequency that is visible or invisible to human eyes.

At block 508, electromagnetic radiation within the predetermined frequency range that is reflected from or transmitted into the patient's skin may be detected or otherwise sensed, e.g., by one or more electromagnetic or optical sensors (e.g., 210, 310, 410). If the electromagnetic radiation emitted at block 504 was modulated at block 506, then at block 510, the information carried in the modulated electromagnetic radiation may be detected and/or provided to logic, such as integral logic 212 or to logic external to the support structure via communication interface 314. At block 512, a pulse rate of the patient may be determined based at least in part on the electromagnetic radiation detected at block 508. If the detected electromagnetic radiation was modulated to carry information at block 506, then at block 514, electromagnetic radiation that (i) has a wavelength that is within the predetermined frequency range described above, and (ii) does not carry the information, may be excluded from the determination of block 512. Consequently, ambient light that happens to be within the predetermined frequency range may not interfere (e.g., as noise) with detecting the patient's pulse.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope

1. An apparatus to aid in cardiopulmonary resuscitation, CPR, comprising:
a support structure shaped to support a head and neck of a patient at respective positions suitable for opening an airflow of the patient during performance of CPR on the patient;
one or more electromagnetic radiation sources mounted within recesses of the support structure or on a surface of the support structure, at which the one or more electromagnetic radiation sources do not come into physical contact with the patient's skin when the patient's head and neck are properly supported by the structure, to emit electromagnetic radiation having a wavelength within a predetermined frequency range of wavelengths onto skin of the patient; and
one or more electromagnetic or optical sensors mounted within recesses of the support structure or on a surface of the support structure, at which the one or more electromagnetic or optical sensors do not come into physical contact with the patient's skin when the patient's head and neck are properly supported by the structure, to detect electromagnetic radiation within the predetermined frequency range that is reflected from or transmitted into the patient's skin, wherein the detected electromagnetic radiation is for use in determining one or more vital signs of the patient.

2. (canceled)

3. (canceled)

4. The apparatus of claim 1, wherein the one or more electromagnetic or optical sensors are mounted at positions on a surface of the support structure that do not come into physical contact with the patient's skin when the patient's head and neck are properly supported by the support structure.

5. The apparatus of claim 1, wherein the one or more electromagnetic or optical sensors are mounted within a neck cradle of the support structure.

6. The apparatus of claim 5, wherein the one or more electromagnetic or optical sensors are mounted at one or more sides of the neck cradle.

7. The apparatus of claim 1, wherein the one or more electromagnetic radiation sources are mounted on the support structure in a manner selected to avoid physical contact with the patient's skin.

8. The apparatus of claim 1, wherein at least one of the one or more electromagnetic radiation sources surrounds at least one of the one or more electromagnetic or optical sensors.

9. The apparatus of claim 1, wherein the one or more electromagnetic radiation sources are spaced from the one or more electromagnetic or optical sensors.

10. The apparatus of claim 1, wherein the electromagnetic or optical sensors emit modulated electromagnetic radiation.

11. The apparatus of claim 1, further comprising one or more pressure sensors and a logic operably coupled with the one or more electromagnetic or optical sensors and the one or more pressure sensors, wherein the logic is configured to determine the one or more vital signs of the patient based on signals from both the one or more electromagnetic or optical sensors and the one or more pressure sensors.

12. A method of detecting return of spontaneous circulation ROSC during performance of cardiopulmonary resuscitation, CPR, comprising:
resting a patient's neck on a support structure shaped to support the patient's head and neck at respective positions suitable for opening an airflow of the patient during performance of CPR on the patient;
causing one or more electromagnetic radiation sources mounted within recesses of the support structure or on a surface of the support structure, at which the one or more electromagnetic radiation sources do not come into physical contact with the patient's skin when the patient's head and neck are properly supported by the structure, to emit electromagnetic radiation having a wavelength within a predetermined frequency range onto skin of the patient;
detecting, using one or more electromagnetic or optical sensors mounted within recesses of the support structure or on a surface of the support structure, at which the one or more electromagnetic or optical sensors do not come into physical contact with the patient's skin when the patient's head and neck are properly supported by the structure, electromagnetic radiation within the predetermined frequency range that is reflected from or transmitted into the patient's skin; and
determining one or more vital signs of the patient based on the detected electromagnetic radiation.

13. The method of claim 12, further comprising modulating electromagnetic radiation emitted by the one or more electromagnetic radiation sources to carry information.

14. The method of claim 13, further comprising detecting, by the one or more electromagnetic or optical sensors, the information carried in the modulated electromagnetic radiation.

15. The method of claim 14, further comprising excluding, from the determination of one or more vital signs of the patient, any electromagnetic radiation having a wavelength within the predetermined frequency range that is not modulated to carry the information.
